(19) United States
(12) Patent Application Publication
(10) Pub. No.: US 2026/0295134 A1
(43) Pub. Date: Oct. 1, 2026

(54) CONTROLLING AN APPARATUS FOR PRODUCTION OF MEDICAL FLUID FOR USE IN DIALYSIS THERAPY

(71) Applicant: GAMBRO LUNDIA AB, Lund (SE)

(72) Inventors: Christian VARTIA, Veberöd (SE); Vishnu SHYAM, Lund (SE); Arash DARBANDI, Oak Brook, IL (US)

(21) Appl. No.: 19/477,705

(22) PCT Filed: Apr. 25, 2024
(86) PCT No.: PCT/EP2024/061350

§ 371 Date: Oct. 22, 2025

(30) Foreign Application Priority Data
Apr. 25, 2023 (SE) 2350494-7

(51) Int. Cl. A61M 1/16; A61M 1/26; B01F 23/40; B01F 35/21; B01F 35/22; B01F 101/22; G16H 40/63

(52) U.S. Cl. CPC A61M 1/1656; A61M 1/1607; A61M 1/267; B01F 23/40; B01F 35/2113; B01F 35/2115; B01F 35/2132; B01F 35/2133; B01F 35/2135; B01F 35/2202; G16H 40/63; A61M 2205/50; B01F 2101/22

(57) ABSTRACT

A fluid preparation apparatus is configured to provide (205) medical fluid for use in dialysis therapy, by generating (204) a mixture of a base fluid and one or more concentrate fluids, each of which is stored in a respective container. A control device determines (203) a reference concentration of the concentrate fluid(s) at a reference time point, by operating (202) the apparatus to pump the concentrate fluid(s) to a sensor device for measuring a composition-related parameter. The control device estimates (206) a momentary concentration of the concentrate fluid(s) as a function of time, by use of the reference concentration and at least one calculation function for calculation of evaporative loss of water from the container(s) over time. Before or during production of the mixture, the control device adjusts (207) a mixing ratio between the base fluid and the concentrate fluid(s) based on the momentary concentration, to produce the mixture with a target composition.

### TECHNICAL FIELD

The present disclosure relates generally to dialysis therapy, and in particular to a technique of producing a medical fluid for use by a dialysis system when performing dialysis therapy.

### BACKGROUND ART

Dialysis therapy is undertaken to replace or supplement the normal blood-filtering function of the kidneys. It is used when the kidneys are not working well, which is known as kidney failure and includes acute kidney injury (AKI) and chronic kidney disease (CKD). Dialysis therapy involves removal of water from the body of the patient suffering from kidney failure, as well as exchange of solutes with the patient's blood. One example of dialysis therapy is extracorporeal (EC) blood therapy, in which blood is circulated outside of the patient and interfaced with one or more medical fluids. Modalities of extracorporeal blood therapy include hemodialysis (HD), hemofiltration (HF) and hemodiafiltration (HDF). Another example of dialysis therapy is peritoneal dialysis (PD), in which a medical fluid is infused into the peritoneal cavity of the patient to interface with the blood of the patient through the peritoneal membrane.

Medical fluids used in HD and PD are commonly known as dialysis fluids. In HF, the medical fluid is known as replacement fluid, since it is infused into the blood of the patient to replace fluid removed during therapy. In HDF, both dialysis fluid and replacement fluid are used.

Extracorporeal blood therapy by HD, HF or HDF is performed differently for treatment of patients with AKI compared to patients with CKD, by use of a different type of dialysis machine. Generally, compared to CKD patients, AKI patients are treated continuously over a longer period of time and at lower fluid flow rates. Such continuous treatment is commonly known as CRRT (Continuous Renal Replacement Therapy).

PD may be performed manually or be automated. In automated peritoneal dialysis (APD), the dialysis treatment is controlled by a machine, commonly known as a “cycler”. The machine is connected in fluid communication with the peritoneal cavity and is operated to control the flow of fresh dialysis fluid into the peritoneal cavity and the flow of spent dialysis fluid from the peritoneal cavity.

Over time, dialysis therapy consumes large quantities of medical fluid. In some modalities of dialysis therapy, pre-made (ready-made) medical fluid is delivered in prefilled bags to the point of care. For example, conventional PD is performed by use of prefilled bags. AKI machines are configured to use prefilled bags of medical fluid, by staff installing a prefilled bag before treatment and replacing the prefilled bag as required. On the other hand, CKD machines have integrated capability to generate medical fluid on-demand by mixing one or more concentrate fluids with water, so-called on-line fluid generation. Recently, PD machines with integrated capability of on-line fluid generation have been proposed. The concentrate fluids are supplied in prefilled bags, which are significantly smaller than the prefilled bags of medical fluid.

Local production of medical fluid at the point-of-care is attractive since it reduces the cost and environmental impact of transporting large amounts of ready-made fluid and the burden of storing and handling the heavy bags of ready-made fluid.

The fluid in a prefilled bag for use in dialysis therapy has a nominal composition, which needs to be met within predefined tolerances. During storage of pre-filled bags, water may evaporate from the fluid inside the bags, through the wall material of the bag, causing the concentration of the fluid to increase over time. Concentrate fluids are more sensitive to evaporative water loss than ready-made fluids, because of their smaller water content.

It is generally desirable to relax the concentration tolerances for concentrate fluids in prefilled bags for use in production of medical fluid, for example to increase the useful life of the concentrate fluids and/or to reduce the requirements on the manufacturing of the concentrate fluids.

### SUMMARY

It is an objective to at least partly overcome one or more limitations of the prior art.

One objective is to provide a technique of relaxing the tolerances for the concentration of concentrate fluids in prefilled bags for use in production of medical fluid.

One or more of these objectives, as well as further objectives that may appear from the description below, are at least partly achieved by a control device, an apparatus for producing medical fluid, a method, and a computer-implemented method according to the independent claims, embodiments thereof being defined by the dependent claims.

A first aspect is a control device for operating an apparatus for producing a medical fluid for use in dialysis therapy. The control device is configured to operate, in a respective production phase, the apparatus to generate a mixture of a base fluid and one or more concentrate fluids, and produce the medical fluid based on the mixture, wherein each of the one or more concentrate fluids is stored in a respective container. The control device being further configured to: operate the apparatus, in an initialization phase, to pump the one or more concentrate fluids to a sensor device for measuring a composition-related parameter; determine, based on an output signal of the sensor device during the initialization phase, a reference concentration of the respective concentrate fluid at a reference time point; and estimate, by use of the reference concentration and at least one calculation function for calculation of evaporative loss of water from the respective container over time, a momentary concentration of the respective concentrate fluid as a function of time. The control device is configured to, at one or more time points before or during the respective production phase and based on the momentary concentration, adjust a mixing ratio between the base fluid and the one or more concentrate fluids in the mixture so as to produce the mixture with a target composition.

A second aspect is an apparatus for producing a medical fluid for use in dialysis therapy. The apparatus comprises: an inlet for a base fluid; an inlet for a respective concentrate fluid; a mixing arrangement operable to mix the base fluid with the respective concentration fluid; a fluid distribution arrangement operable to selectively supply the base fluid and the respective concentration fluid to the mixing arrangement; a sensor device for measuring a composition-related parameter; and a control device according to the first aspect.

A third aspect is a computer-implemented method of operating an apparatus to produce a medical fluid for use in dialysis therapy by mixing a base fluid with one or more concentrate fluids, wherein each of the one or more concentrate fluids is stored in a respective container. The method comprises: operating the apparatus, in an initialization phase, to pump the one or more concentrate fluids to a sensor device for measuring a composition-related parameter; determining, based on an output signal of the sensor device during the initialization phase, a reference concentration of the respective concentrate fluid at a reference time point; operating, in a respective production phase, the apparatus to generate a mixture of a base fluid and one or more concentrate fluids at a mixture ratio and to produce the medical fluid based on the mixture; estimating, by use of the reference concentration and at least one calculation function for calculation of evaporative loss of water from the respective container over time, a momentary concentration of the respective concentrate fluid as a function of time; and adjusting, at one or more time points before or during the respective production phase and based on the momentary concentration, the mixing ratio between the base fluid and the one or more concentrate fluids in the mixture so as to produce the mixture with a target composition.

A fourth aspect is a computer-readable medium comprising instructions, which when executed by a processor in the control device of the first aspect, causes the control device to perform the method of the third aspect.

These aspects provide a technique of operating an apparatus to produce medical fluid. The apparatus produces the medical fluid during a production phase. The medical fluid may be produced during a single production phase of the apparatus, or repetitively during a sequence of time-separated production phases. During the respective production phase, the apparatus is operated to generate a mixture of a base fluid and one or more concentrate fluids. The target composition of the mixture may be the same or differ between production phases, and may even vary during a production phase. The foregoing aspects provide a technique of compensating for evaporative water loss from a concentrate fluid that is stored in a container and used by the apparatus for producing the medical fluid. The technique involves an initialization phase, in which the apparatus is operated for determination of a reference concentration of the concentrate fluid at a reference time point, and also involves using a calculation function for estimating evaporative water loss over time. The calculation function, which may be predefined and optionally updated over time, is used for estimating a momentary concentration of the concentrate fluid in the container, given the reference concentration at the reference time point. Through knowledge about the momentary concentration, it is possible to adjust the mixing ratio between the base fluid and the concentrate fluid to attain a target composition of the resulting mixture. This adjustment thus compensates, at least partly, for evaporative water loss from the container over time and makes it possible to use the concentrate fluid in the container for production of medical fluid over an extended period of time. The technique relaxes the tolerances for the concentration of the concentrate fluid in the container, through the measurement of the reference concentration and the estimation of evaporative water loss based thereon. The technique also relaxes the need to perform measurements of the momentary concentration of the concentrate fluid in the container subsequent to the initialization phase, which in turn limits expenditure of concentrate fluid and saves time.

Below, some embodiments of the control device of the first aspect are recited. These embodiments are also applicable to the second to fourth aspects.

In some embodiments, the control device is configured to calculate, by use of the at least one calculation function, an aggregation of the evaporative loss of water from the respective container, said aggregation being made from the reference time point to a selected time point, and the control device is further configured to estimate the momentary concentration of the respective concentrate fluid at the selected time point by compensating the reference concentration of the respective concentrate fluid by the aggregation of the evaporative water loss from the respective container.

In some embodiments, the control device is configured to determine a momentary concentration change of the respective concentrate fluid based on a momentary change in evaporative loss given by the at least one calculation function and based on a momentary amount of the respective concentrate fluid in the respective container over time, and to estimate the momentary concentration of the respective fluid at the selected time point by calculating an aggregated value of the momentary concentration change from the reference time point to the selected time point, and adding the aggregated 10 value from the reference concentration.

In some embodiments, the control device is further configured to perform a validation procedure, which comprises: operating the apparatus to pump at least one concentrate fluid among the one or more concentrate fluids to the sensor device; and determining, based on an output signal of the sensor device an updated concentration of the at least one concentrate fluid; and the control device is further configured to update the at least one calculation function based on the reference concentration of the at least one concentrate fluid, the updated concentration of the at least one concentrate fluid, the reference time point and a validation time point associated with the validation procedure.

In some embodiments, the one or more concentrate fluids comprises a first concentrate fluid in a first container and a second concentrate fluid in a second container.

In some embodiments, the control device is configured to, during the initialization phase: operate the apparatus to generate a first test mixture of the base fluid and the first concentrate fluid, at a first mixing ratio, and pump the first test mixture to the sensor device; obtain a first sensor value for the first test mixture from the output signal of the sensor device; operate the apparatus to generate a second test mixture of at least the base fluid and the second concentrate fluid, at a second mixing ratio, and pump the second test mixture to the sensor device; and obtain a second sensor value for the second test mixture from the output signal of the sensor device.

In some embodiments, the control device is configured to determine, based on the first and second mixing ratios and the first and second sensor values, the reference concentration of the first concentrate fluid and the reference concentration of the second concentrate fluid.

In some embodiments, the at least one calculation function comprises a first calculation function for the first container, and the control device is further configured to perform a validation procedure, wherein the validation procedure comprises: operating the apparatus to generate a further mixture of the base fluid and the first concentrate fluid, at a further mixing ratio, and pump the further mixture to the sensor device; obtaining a further sensor value for the further mixture from the output signal of the sensor device; and determining, based on the further sensor value and the further mixing ratio, an updated concentration of the first concentrate fluid; and the control device is further configured to update the first calculation function based on the reference concentration of the first concentrate fluid, the updated concentration of the first concentrate fluid, the reference time point and a validation time point associated with the validation procedure.

In some embodiments, the control device is configured to update the first calculation function by adjusting one or more parameters of the first calculation function so that that the first calculation function results in an aggregated water loss from the reference time point to the validation time point that matches a difference in water content between the reference concentration of the first concentrate fluid and the updated concentration of the first concentrate fluid.

In some embodiments, the control device is configured to, before or during the respective production phase after the validation procedure, estimate the momentary concentration of the first concentrate fluid in the first container as a function of time by use of the thus-updated first calculation function.

In some embodiments, the control device is configured to, before or during the respective production phase after the validation procedure, estimate the momentary concentration of the second concentrate fluid in the second container as a function of time by use of the thus-updated first calculation function.

In some embodiments, the first concentrate fluid comprises one or more electrolytes, and the second concentrate fluid comprises an osmotic agent.

In some embodiments, the reference concentration and the momentary concentration represent the concentration of a representative solute in the respective concentrate fluid.

In some embodiments, the base fluid is purified water.

In some embodiments, the at least one calculation function is configured to quantify a momentary evaporative loss of water from the respective container over time.

In some embodiments, the control device is configured to convert the momentary evaporative loss of water from the respective container over time into the momentary concentration of the respective concentrate fluid as a function of time, based on the reference concentration of the respective concentrate fluid and based on a momentary amount of the respective concentrate fluid in the respective container over time.

In some embodiments, the at least one calculation function is an analytical function or an empirical function.

In some embodiments, the control device is configured to determine the amount of concentrate fluid within the respective container over time during the respective production phase.

In some embodiments, the calculation function is a function of at least one of: a physical property of the respective container, a humidity in ambient air at the respective container, a temperature of the concentrate fluid within the respective container, a partial vapor pressure of water in the concentrate fluid within the respective container, or an amount of concentrate fluid in the respective container.

In some embodiments, the composition-related parameter represents electrical conductivity.

Still other objectives, aspects, embodiments, and technical effects, as well as features and advantages may appear from the following detailed description, from the attached claims as well as from the drawings.

### BRIEF DESCRIPTION OF THE DRAWINGS

FIGS. 1A-1B are schematic views of dialysis systems that include a fluid preparation apparatus, and FIG. 1C is a block diagram of an example fluid preparation apparatus (FPA).

FIG. 2A is a flow chart of an example method of operating an FPA, and FIGS. 2B-2C are flow charts of example procedures for use in the method of FIG. 2A.

FIG. 3 is a graph of measured conductivity during an initialization procedure in accordance with FIG. 2B.

FIG. 4 is a graph of concentration values determined in a method in accordance with FIG. 2A, and corresponding concentration calculation functions (CCFs) for use by the method.

FIG. 5 is a graph of evaporative water loss over time from two fluid bags containing electrolytes and glucose, respectively.

FIG. 6 shows time sequences of events during four consecutive sessions of PD therapy, including events performed by an FPA in accordance with embodiments.

FIG. 7A is a schematic diagram of an example FPA for on-demand generation of medical fluid, and FIG. 7B is a schematic diagram of an example FPA for batch-wise generation of medical fluid.

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more”, even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

As used herein, the terms “multiple”, “plural” and “plurality” are intended to imply provision of two or more elements, whereas the term a “set” of elements is intended to imply a provision of one or more elements. The term “and/or” includes any and all combinations of one or more of the associated listed elements.

It will furthermore be understood that although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure.

Well-known functions or constructions may not be described in detail for brevity and/or clarity. Unless otherwise defined, all terms (including technical and scientific terms) used herein have the same meaning as commonly understood by one of ordinary skill in the art to which this disclosure belongs.

As used herein, “dialysis therapy” refers to any therapy that replaces or supplements the renal function of a patient by use of a medical fluid. Dialysis therapy includes, without limitation, extracorporeal blood therapy and peritoneal dialysis therapy.

As used herein, “medical fluid” refers to any fluid that is consumed as a result of dialysis therapy. Medical fluid includes, without limitation, dialysis fluid for infusion into the peritoneal cavity during peritoneal dialysis therapy, dialysis fluid for supply to a dialyzer during EC blood therapy, replacement fluid and substitution fluid for infusion into blood during EC blood therapy, priming fluid, and fluid for disinfection and/or cleaning of the dialysis system.

As used herein, a “concentrate fluid” or “concentrate” is a water-containing liquid that contains one or more compounds (“solutes”) at a concentration that is higher than at the final use of the liquid. Thus, a concentrate is produced to be diluted by a solvent, also denoted “base fluid” herein. For example, the base fluid may be water or another water-containing concentrate.

Like reference signs refer to like elements throughout.

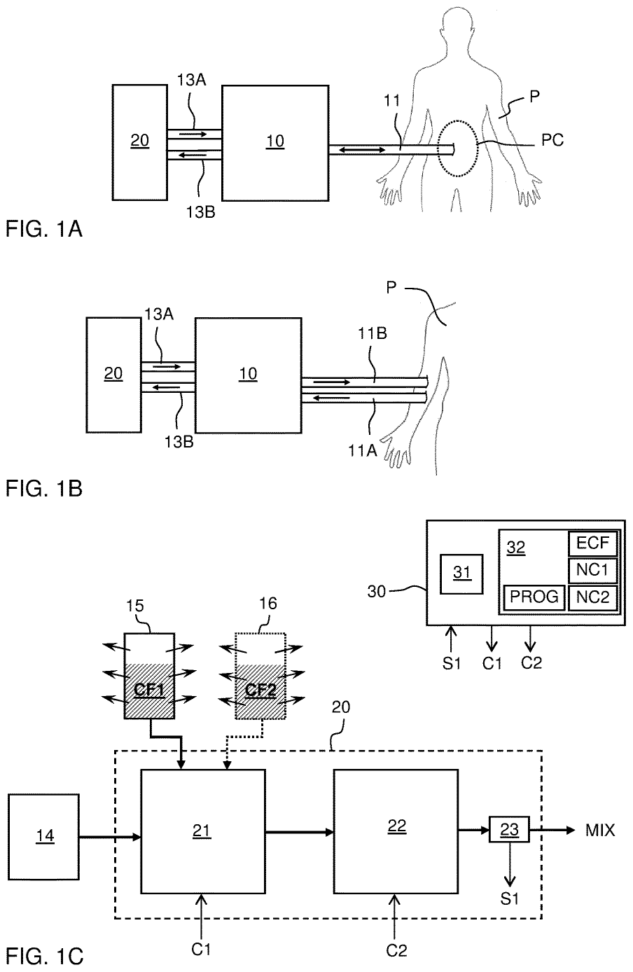

The present disclosure relates to a technique of generating medical fluid for use by a dialysis system. The technique is applicable to both peritoneal dialysis (PD) therapy and extracorporeal (EC) blood therapy. For context only, fluid generation (preparation) in relation to PD therapy and EC blood therapy will be briefly discussed with reference to FIGS. 1A-1B.

FIG. 1A is a generic overview of a dialysis system for PD therapy. The dialysis system comprises a therapy system 10, which is fluidly connected to the peritoneal cavity PC of a patient P. As indicated by a double-ended arrow, the therapy system 10 is operable to convey fresh dialysis fluid into PC and to receive spent dialysis fluid from PC on a fluid path 11. The fluid path 11 may be defined by tubing that connects to an implanted catheter (not shown) in fluid communication with the peritoneal cavity PC. The therapy system 10 may be configured for any type of PD therapy. In one example, the therapy system 10 comprises one or more containers that are manually handled to perform PD therapy. In another example, the therapy system 10 comprises a dialysis machine (“cycler”) that performs the PD therapy. The dialysis system further comprises a fluid preparation apparatus, FPA, 20, which is configured to generate dialysis fluid for use by the therapy system 10. The dialysis fluid is supplied from the FPA 20 to the therapy system 10 on a first fluid path 13A. PD therapy is typically implemented as daily treatment sessions, each comprising a number of fluid exchange cycles. Each fluid exchange cycle consists of a fill phase, a dwell phase and a drain phase, performed in sequence. In the fill phase (FP), fresh dialysis fluid is supplied to PC on fluid path 11. In the dwell phase (DWP), the dialysis fluid resides in PC. In the drain phase (DP), spent dialysis fluid is extracted from PC on fluid path 11. The spent dialysis fluid may be returned to the FPA 20 on a second fluid path 13B, as shown, or be directed to a drain.

FIG. 1B is a generic overview of a dialysis system for EC blood therapy. The dialysis system comprises a therapy system 10, which is fluidly connected to the vascular system of a patient P on a fluid path. In the illustrated example, the fluid path is defined by tubing 11A for blood extraction and tubing 11B for blood return. As indicated by arrows, the therapy system 10 is operable to draw blood from the patient P through tubing 11A, process the blood, and return the processed blood to the patient through tubing 11B. The tubing 11A, 11B is connected to an access device (for example a catheter, graph or fistula, not shown) in fluid communication with the vascular system of the patient P. The therapy system 10 may be configured to process the blood by any form of EC blood therapy, such as HD, HF or HDF. In such therapy, medical fluid in the form of dialysis fluid and/or replacement fluid is consumed. The medical fluid is supplied from an FPA 20 to the therapy system 10 on the first fluid path 13A. The spent dialysis fluid may be returned to the FPA 20 on a second fluid path 13B, as shown, or be directed to a drain.

FIG. 1C is a block diagram of an example fluid preparation apparatus, FPA, 20. The FPA 20 is operable to produce and supply an output fluid, MIX, which may be a medical fluid for use by a therapy system, for example as shown in FIGS. 1A-1B. Alternatively, MIX may be an intermediate fluid which is adapted to be processed into such a medical fluid, for example by admixing of one or more further ingredients in solid or liquid form. The FPA 20 comprises a fluid distribution arrangement 21, which is fluidly connected to a source 14 for a base fluid, as well one or more containers (two shown) 15, 16 that hold a respective concentrate fluid (“concentrate”) CF1, CF2. The containers may be physically separated, as shown, or be separate compartments in a unitary structure. In some embodiments, the base fluid is water, typically with a sufficient purity to comply with quality requirements for water to be included in dialysis fluid, for example according to ISO 23500-3 (“Water for haemodialysis and related therapies”, 2019). However, any appropriate liquid may be used as base fluid, including but not limited to a saline solution. The source 14 may be a centralized source, which is arranged to supply base fluid to a plurality of FPAs 20, or a dedicated source for a single FPA 20.

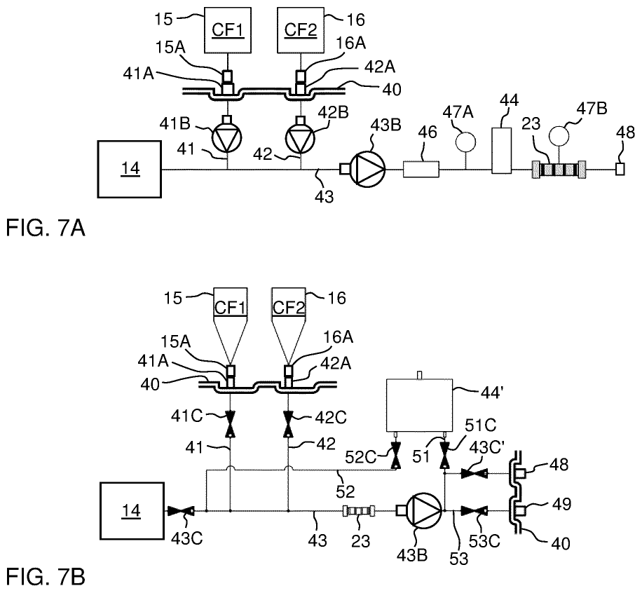

The fluid distribution arrangement 21 is operable to receive and distribute the base fluid and the concentrate(s) to a downstream mixing arrangement 22, which is operable to mix the base fluid and the concentrate(s). The fluid distribution arrangement 21 has any suitable configuration and may comprise a fluid manifold, one or more pumps, one or more valves, etc. The mixing arrangement 22 is also of conventional design and may comprise one or more mixing devices, one or more pumps, one or more valves, etc. The present disclosure is not limited to any particular configuration of the fluid distribution arrangement 21 or the mixing arrangement 22. Depending on implementation, the FPA 20 may be configured to produce the output fluid, MIX, in either discrete batches or on-demand. Detailed examples of structures for on-demand production and batch-wise production are given further below with reference to FIGS. 7A-7B.

The containers 15, 16 are configured to be releasably attached to the FPA 20, to enable replacement of the respective container for a new prefilled container. The containers 15, 16 may be replaced individually or jointly. In one example, new containers 15, 16 are attached to the FPA 20 after a given number of treatment sessions. In another example, each container 15, 16 is replaced whenever it is deemed to be in need of replacement, for example based on the amount of remaining concentrate in the container 15, 16. In the following, the time period when the respective container is attached to the FPA 20 is denoted an “in-use period”.

The FPA 20 includes a sensor device 23, which is located downstream of the mixing arrangement 22 to receive and measure a composition-related parameter (CRP) of the output fluid, MIX, or any other fluid that is directed to or through the sensing device 23. The CRP may represent electrical conductivity, or equivalently electrical resistivity. In a variant, the CRP represents the concentration of a solute in MIX, for example bicarbonate or an electrolyte such as sodium, potassium, calcium, magnesium, chloride, etc. If the medical fluid is generated for use in PD, the solute may alternatively be an osmotic agent such as glucose. In a further alternative, the CRP may represent the concentration of hydrogen ions, for example in the form of a pH value. As shown, the CRP sensor 23 provides a sensor signal S1, which is indicative of the CRP.

A control device or control system 30 is arranged to receive and process the sensor signal S1, and generate and output control signals C1, C2 for controlling the operation of the fluid distribution arrangement 21 and the mixing arrangement 22, respectively. The control signals C1, C2 may be generated in accordance with predefined logic that is implemented by the control device 30. The predefined logic may be implemented by hardware, or a combination of hardware and software. In the example of FIG. 1C, the control device 30 comprises processing circuitry 31 and computer memory 32. The processing circuitry 31 may comprise one or more processors, such as a CPU, DSP, ASIC, FPGA, etc. A control program, PROG, may be stored in the memory 32 and executed by the processing circuitry 31 to perform any of the methods, procedures, or functions as described herein. The control program may be supplied to the control device 30 on a computer-readable medium, which may be a tangible (non-transitory) product (e.g., magnetic medium, optical disk, read-only memory, flash memory, etc.) or a propagating signal. As indicated in FIG. 1C, the memory 32 may also store control data for use by the processing circuitry 31, for example an evaporation calculation function (ECF), and nominal composition data (NC1, NC2) for the concentrates CF1, CF2.

Although not shown in FIG. 1C, the control device 30 may comprise one or more I/O interfaces for input of S1 and output of C1, C2, and optionally for connection to a user interface. The term “user interface” is intended to include any and all devices that are capable of performing guided human-machine interaction comprising presentation of information and receipt of user input. The I/O interface(s) may be configured for wired or wireless data communication.

As described in the Background section, there are many advantages associated with the use of concentrates instead of ready-made fluids. The following description will assume that the FPA 20 is configured to mix two concentrates (CF1, CF2 in FIG. 1C) with purified water (base fluid) to produce a dialysis fluid for use in PD, with CF1 containing the electrolytes for the dialysis fluid to be produced, and CF2 containing the osmotic agent, in this example glucose. In some embodiments, CF1 has a nominal dilution factor of 20, CF2 contains 40%-70% glucose, and the respective container 15, 16 has a volume of 1-5 L, for example 1 L, 3.5 L or 5 L. The respective container 15, 16 may contain concentrate for use during a time period of at least 3-14 days.

In a non-limiting example used in the following calculations, CF2 contains 50% glucose, and the respective container 15, 16 has a volume of 1 L. A typical APD session consumes 12 L of dialysis fluid. Assuming that the dialysis fluid contains 2.27% glucose and is prepared by mixing water, CF1 and CF2, and that the residual volumes of concentrate in the containers 15, 16 are discarded when the APD session is completed, the resulting waste of concentrate is 35% for CF1 and 40% for CF2. Assuming instead that CF1, CF2 in the containers 15, 16 are used for producing dialysis fluid for multiple APD sessions, for example on different days, it can be shown that the resulting waste of concentrate is reduced to 10% for both CF1 and CF2 when each APD session consumes 12 L of the dialysis fluid with 2.27% glucose. Here, it is to be noted that the dialysis fluid is either produced batch-wise before each session or produced on-demand during the respective session. Thus, the production of dialysis fluid from CF1, CF2 is performed over an extended period of time.

Clearly, it would be beneficial to produce medical fluid from CF1, CF2 in the containers 15, 16 for use in multiple sessions. Significant savings would be achieved, both economically and environmentally, through less waste of concentrate and less waste of container material per session. It would also lower the burden on the user since unpacking, placing and connection of containers need not be performed on a daily basis, but may be made every 5-10 days.

The present Applicant has found that evaporation of water from the concentrate containers 15, 16 poses a significant obstacle to the use of CF1, CF2 for producing medical fluid for multiple sessions. During storage, water evaporation from a concentrate container may be partly prevented by covering it by a protective material, known as an “over-pouch”. However, as the over-pouch is removed prior to use, the evaporation rate will increase significantly, as indicated by arrows in FIG. 1C. Since concentration containers are typically made of flexible material, in the form of flexible bags, the shape of the container will change as the concentrate is consumed. Specifically, the volume of the concentrate will decrease faster than the surface area of container material that is available for water diffusion out of the container. This causes the up-concentration rate of the concentrate in the concentrate container to increase over time.

One way to mitigate the problem of evaporative loss would be to produce the concentrate container from a material that is substantially impermeable to water vapor.

However, such materials are significantly more expensive than common plastic materials such as polyvinyl chloride (PVC), polyethylene (PE), polypropylene (PP), etc. Another way of attacking the problem might be to manufacture the container with substantially thicker walls of plastics material, to thereby decrease the rate of evaporation. However, this would increase the environmental impact and cost.

Given the shortcomings of these alternative solutions, the present Applicant has developed a technique of estimating and accounting for the evaporative loss from a concentration container during the in-use period. The novel technique is both more cost-effective and more sustainable than the above-mentioned alternatives.

Further, the novel technique may reduce the requirements on the manufacturing process of the concentrates and/or the container material. The novel technique may also reduce the time to therapy and concentrate waste by reducing the need to intermittently perform concentration tests during the in-use period.

The novel technique uses a calculation function for estimating the evaporation of water from the concentrate in the respective container. In the following, the calculation function is denoted “evaporation calculation function”, or ECF. Specifically, the ECF is configured to quantify the evaporative loss of water from the container and allows for calculation of the concentration of the concentrate in the container as a function of time. The ECF may be an analytical function, which is derived based on a predetermined physical model of the respective container. Such an analytical function may or may not be recursive. An example of a physical model will be given and motivated below. Parameters of the physical model may be determined as tabulated values or be given by preparatory measurements.

In an alternative, the ECF is a purely empirical function. Such a function is given solely by measurement data and is not derived based on a physical model. The ECF may be defined by the functional dependence of a dependent variable (response variable) on one or more independent variables (explanatory variables), and the relationship between the dependent variable and the independent variable(s) may be determined based on measurement data, for example by conventional regression analysis.

In some embodiments, which are applicable to both analytical and empirical ECFs, the ECF depends on at least the volume of the concentrate inside the container at the respective time point (“momentary concentrate volume”). As used herein, “momentary” implies a value at an individual time point. The ECF, whether analytical or empirical, may also depend on one or more additional input variables, including one or more of: the temperature of the concentrate, the humidity of ambient air around the container, or the water vapor pressure (partial vapor pressure) in the concentrate. The additional input variables may be time-dependent and represented by momentary values, or time-invariant. The ECF may also account for one or more physical properties of the container, for example the material of the container, the surface area of the container, etc. The material of the container may be characterized by a structural flexibility, thickness, water vapor permeability, etc.

Provided that the initial concentration of the concentrate is known, estimated or measured, the analytical or empirical ECF allows the evaporative loss to be tracked (calculated) as a function of time and converted into a concentration value of the concentrate as a function of time.

In an example, the concentration change (dc/dt) in the concentrate is assumed to depend on the evaporation rate (dm/dt) and the momentary concentrate volume in the container (V), according to:

$$dc / dt = f 1 ( dm / dt , V ) = dm / dt \cdot 1 / V \qquad ( 1 )$$

In an example, the evaporation rate (dm/dt) is assumed to depend on the effective area for evaporation (A), the partial vapor pressure (p_{w}) of water in the concentrate, the temperature (T) of the concentrate, and the humidity (H) in ambient air at the container, according to:

$$dm / dt = f 2 ( A , {p}_{w} , T , H ) \qquad ( 2 )$$

Here, f2 is an ECF, which may be determined analytically or empirically. In an example of an analytical model, the evaporation rate (dm/dt) is assumed to be proportional to the effective area (A) and the partial vapor pressure (p_{w}), respectively. Further, the dependence of the momentary evaporation rate on the temperature may be given by an Arrhenius equation: dm/dt∝exp(−Ea/R·T), with Ea being the activation energy for evaporation, and R being the universal gas constant. Further, the evaporation rate (dm/dt) may be assumed to be inversely dependent on the humidity (H) in ambient air, for example represented as relative humidity in the surroundings of the container. Under these assumptions, Eq. (2) may be written as:

$$dm / dt = K 1 \cdot A \cdot {p}_{w} \cdot 1 / H \cdot exp ( - K 2 \cdot T ) \qquad ( 3 )$$

with K1, K2 being model parameters that may be determined based on measurement data.

The evaporation rate may be time-dependent, if any one of the effective area, the partial vapor pressure, the temperature, or the relative humidity changes over time.

In one example, the effective area (A) may be assumed to be a function of the momentary concentrate volume (V) in the container. For example, if the container is a flexible bag, the effective area may be approximated by a sphere, A=4π·(3/4·V/π)^{2/3}. The partial vapor pressure of water (p_{w}) in the concentrate may be given by Raoult's law: p_{w}=p_{w,0}·x_{w}, with p_{w,0} being the vapor pressure of pure water, and x_{w} being the mole fraction of water in the concentrate. The temperature of the concentrate (T) may be measured by a temperature sensor associated with the container. The temperature sensor may be arranged in contact with the concentrate or may be no-contact sensor, such as an IR sensor. Alternatively, temperature of the concentrate may be estimated based on the surrounding temperature, if measured. The relative humidity (H) may be measured, for example by hygrometer. Under these assumptions, Eq. (2) may be written as:

$$dm / dt = K {1}^{′} \cdot {V}^{2 / 3} \cdot {x}_{w} \cdot 1 / H \cdot exp ( - K 2 / T ) \qquad ( 4 )$$

which gives the following analytical expression for Eq. (1):

$$dc / dt = K {1}^{″} \cdot {V}^{- 1 / 3} \cdot {x}_{w} \cdot 1 / H \cdot exp ( - K 2 / T ) \qquad ( 5 )$$

Again, K1′, K1″, K2 are model parameters that may be determined based on measurement data.

Each of the input variables (p_{w}, x_{w}, H, T) may or may not be included. If included, any one of the input variables (p_{w}, x_{w}, H, T) may be considered time-dependent or time-invariant, depending on operating conditions and required calculation accuracy. Any input variable that is considered time-invariant may be included as a model parameter in ECF (cf. K1, K1′). It is also conceivable to include further input variables in the ECF.

Irrespective of equation for the concentration change (dc/dt), the concentration of the concentrate in the container at a specific time point, within a calculation period, may be given by a concentration calculation function (CCF):

$$c ( t ) = c 0 + {\int }_{t 0}^{t} ( dc / dt ) dt \qquad ( 6 )$$

with c0 being the concentration at a start time (t0) of the calculation period.

Assuming that Eq. (4) or Eq. (5) is to be evaluated within the calculation period, the momentary concentrate volume (V) may be determined based on a momentary consumption rate of the concentrate (dV/dt), and a reference volume (V0) of concentrate in the container at a given point in time. In some embodiments, the given point in time is the start time (t0), resulting in V (t) being given by:

$$V ( t ) = V 0 + {\int }_{t 0}^{t} ( dV / dt ) dt \qquad ( 7 )$$

It may be noted that the concentrate volume in the container also changes as a result of the evaporation. However, this effect is typically small during the calculation period and may be omitted from the predefined model to simplify the calculations. Depending on implementation, the reference volume (V0) may be a nominal value, calculated value or measured value. The momentary consumption rate (dV/dt) may be measured by a flow meter in a flow path downstream of the container or calculated based on the number of pumping strokes by a volumetric pump, which is arranged to pump the concentrate from the container.

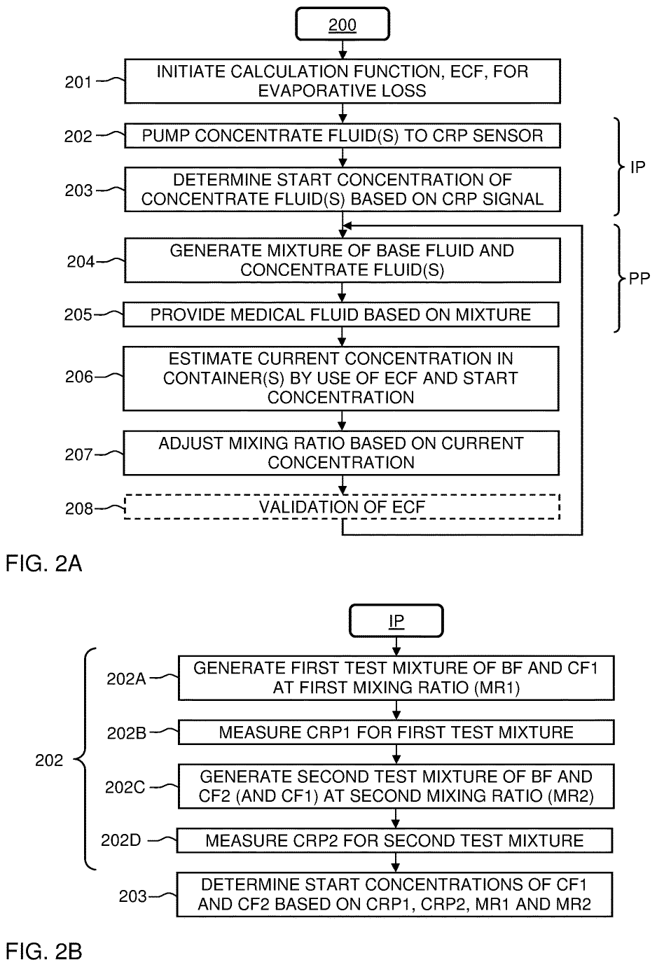

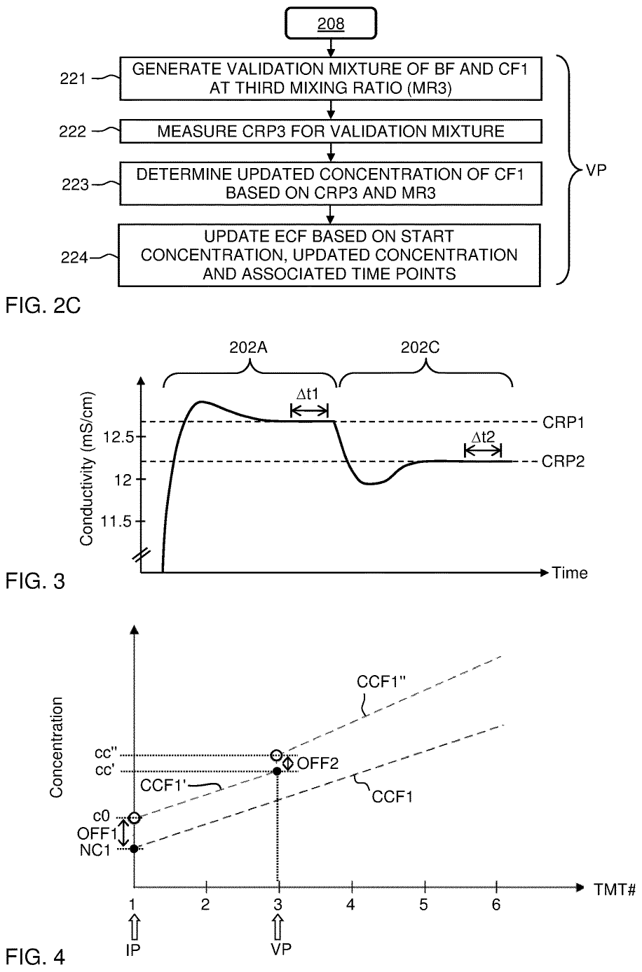

FIG. 2 is a flow chart of an example method 200 for operating an FPA to produce medical fluid for use in dialysis therapy. The method 200 may be performed by the control device 30 in FIG. 1C, through generation of appropriate control signals C1, C2 for the FPA 20. The following description presumes that the medical fluid is generated by mixing the base fluid with CF1 and CF2. However, the description is equally applicable to a smaller or larger number of concentrates.

The method 200 comprises an initialization phase (IP), in which the control device is prepared for concentration calculations, and one or more production phases (PP), in which the FPA is operated to generate the medical fluid by use of the concentration calculations. The IP may be triggered by a predefined event, for example that one of the containers 15, 16 on the FPA 20 is replaced for a new container. The concentration calculations are made separately for CF1 in container 15, and CF2 in container 16.

In the example of FIG. 2A, the method 200 starts by a step 201 of initiating at least one ECF for use in the concentration calculations. The initiating step 201 involves obtaining and/or otherwise preparing at least one EFC for use in the method 200. In a non-limiting example, the at least one ECF is retrieved from memory 32 in step 201. The at least one ECF may be predefined, or stored in the memory during a preceding execution of the method 200. In some embodiments, the at least one ECF is retrieved as part of a concentration calculation function (CCF), for example Eq. (5) above. Step 201 may also allow a caretaker to input one or more customized values of model parameters and/or fixed values of input variables. Depending on implementation, the method 200 may use one ECF for each container 15, 16 or a single ECF for both containers 15, 16.

The IP comprises a step 202 of operating the FPA 20 to pump the concentrates CF1, CF2 to the CRP sensor 23, and a step 203 of determining a reference concentration of the respective concentrate based on the sensor signal S1 from the CRP sensor 23 during step 202. The reference concentration may correspond to c in Eq. (5) and is denoted “start concentration” in the following. Step 202 may be performed in different ways depending on the type and number of concentrates. In a first embodiment, each concentrate is separately pumped to the CRP sensor, for measurement of a CRP value of the respective concentrate CF1, CF2. In a second embodiment, different test mixtures of the base fluid and concentrates CF1, CF1 are pumped to the CRP sensor, for measurement of a CRP value of the respective test mixture. An example of the second embodiment is described further below with reference to FIG. 2B.

The skilled person realizes that step 203 may involve a conversion of a measured CRP value into a concentration value of a representative solute, whether the measured CRP value is a concentration value for another solute, or conductivity. The conversion may be performed based on predefined calibration data for the CRP sensor 20 and the specific concentrates CF1, CF2 that are used. The skilled person readily understands how such a conversion is performed.

In FIG. 2A, the respective production phase (PP) comprises steps 204-205. As indicated by a looping arrow in FIG. 2A, the method 200 may perform a plurality of production phases, separated by a period without production of medical fluid. In step 204, the FPA 20 is operated to mix the base fluid with CF1, CF2 in proportions that are given by the IP, to yield the output fluid, MIX. The proportions correspond to a mixing ratio between the base fluid, CF1 and CF2. In one example, the proportions are calculated based on the start concentration of the respective concentrate, to yield a target composition of MIX. In another example, step 202 comprises generating a test mixture with the target composition, to yield a target CRP value at the CRP sensor 20, and this test mixture is then generated also in step 204 to produce MIX. In step 205, the FPA 20 is operated to provide the medical fluid based on MIX. If MIX is an intermediate fluid, the FPA 20 is operated in step 205 to further process MIX into the medical fluid, before supplying the medical fluid for use by the therapy system (10 in FIGS. 1A-1B). If MIX is the medical fluid, the FPA 20 is operated to output the medical fluid in step 205.

The method 200 further comprises steps 206-207, which may be performed between production periods, as shown, and/or during the respective production period. In step 206, a momentary (“current”) concentration of the respective concentrate is estimated as a function of time during PP by use of the start concentration from step 203 and the ECF(s). The current concentration may be calculated in accordance with Eq. (5). The current concentration is abbreviated cc herein and is given by c (t) at a current time point (tc). Step 206 may also involve determining the momentary (“current”) amount (V) of concentrate in the respective container as a function of time during PP, for example in accordance with Eq. (6), and using the current amount as input data for calculating the current concentration (cc). Depending on implementation, step 206 may also involve measuring or otherwise estimating one or more of the input variables (p_{w}, x_{w}, H, T) for use in calculation the current concentration.

Step 206 may be seen to involve a calculation of the aggregated evaporative loss of water (“aggregated loss”) from the concentrate in the respective container during a calculation period, which extends from a reference time point to a current time point. In some embodiments, the reference time point is the above-mentioned start time (t0). In one non-limiting examples, the reference time point may be when the respective container is installed on the FPA 20, a time during the IP, or when the first PP is started. It is possible that the calculations for different containers use different reference time points. The aggregated loss is an estimation of the total amount of water that has escaped through the wall material of the container during the calculation period.

Further, step 206 may be seen to involve a determination of the current concentration (cc) at the current time point, by compensating the start concentration for the aggregated loss. This adjustment also accounts for the change in the amount of concentrate in the container during the calculation period. The calculation of aggregated loss and the adjustment need not be performed as separate steps but may be merged, for example as shown by Eq. (1)-(6) above.

In some embodiments, step 206 is performed to continuously estimate the current concentration (cc) at consecutive time steps after the reference time point, and step 207 is intermittently performed to use a value of the current concentration from step 206. This has been found to reduce the computational load, since it allows for incremental calculation of the current amount (V) of concentrate in the container and incremental calculation of the current concentration (cc) based on the current amount. In other words, both the current amount (V) and the current concentration (cc) may be continuously tracked throughout execution of the method 200.

In step 207, the mixing ratio between the base fluid, CF1 and CF2 is adjusted based on the current concentration (cc) at one or more time points, to produce MIX with the target composition. In some embodiments, step 207 is performed before start of each production phase (PP) after the first production phase. In some embodiments, step 207 is performed at a predefined time interval during one or more production periods. For example, step 207 may be performed every 15-180 minutes. The time interval may be defined based on the expected impact of evaporative loss on the concentration of the concentrate, to achieve a predefined accuracy of the target composition of MIX.

The target composition of MIX during PP may be a fixed value or a time-varying value. The time-varying value may be entered to the control device 30 before start of dialysis therapy, for example by a caretaker, in the form of a time profile for the target composition during PP. Alternatively or additionally, the caretaker may decide to change the target composition of the medical fluid during on-going therapy.

FIG. 2B is a flow chart of an example of the initialization phase (IP) for determining the start concentrations of the concentrates CF1, CF2 in FIG. 1C, by use of test mixtures. In step 202A, the FPA 20 is operated to generate a first test mixture of the base fluid and CF1 at a first predefined mixing ratio, MR1, and pump the first test mixture to the CRP sensor 23. In step 202B, the control device 30 obtains a first sensor value, CRP1, for the first test mixture from the sensor signal S1. In step 202C, the FPA 20 is operated to generate a second test mixture of the base fluid, CF1 and CF2 at a second predefined mixing ratio, MR2, and pump the second test mixture to the CRP sensor 23. In step 202D, the control device 30 obtains a second sensor value, CRP2, for the second test mixture from the sensor signal S1. In step 203, the start concentrations of CF1 and CF2 are determined based on CRP1, CRP2, MR1 and MR2. The calculations in step 203 are not presented in detail, since they should be readily apparent to the skilled person.

In the example of FIG. 2B, the second test mixture includes both CF1 and CF2, whereas the first test mixture includes only CF1. Thus, compared to CRP1, CRP2 includes the added effect of CF2. It may be noted that the proportion between CF1 and the base fluid may or may not be the same in the first and second test mixtures. Step 203 may be facilitated if the proportion remains the same. If further concentrates are mixed with the base fluid to generate MIX, IP may include a further set of steps, corresponding to steps 202C, 202D, for each such concentrate. Each such further set of steps adds a respective further concentrate to the latest test mixture, at a predefined mixing ratio, and measures a CRP value for the current test mixture.

The example of FIG. 2B has the benefit of allowing the CRP sensor 23 to have a relative narrow detection range of CRP values, since the first and second test mixtures may be defined to yield CRP values that are relatively close to each other and to the CRP value of the output fluid, MIX. This relaxes the performance requirements of the CRP sensor 23 compared to when the CRP sensor 23 is used for measuring a CRP value for the respective concentrate as such.

Another benefit is that the start concentration of CF2 may be determined even if the CRP value of CF2 is small, as long as CF2 has an inherent conductivity-modifying effect when added to a mixture of base fluid and CF1. For example, glucose is known to inherently decrease the conductivity of low-viscosity ionic solutions. This effect has been attributed to an increase in viscosity caused by the added glucose, for example in the article “Electrical conductivity of viscous liquid foods”, by Subbiah B. and Morison K. R, published in Journal of Food Engineering 237, pp. 177-182 (2018). By including CF2 in the second test mixture and not in the first test mixture, the effect of CF2 may be seen in CRP2 compared to CRP1. For example, consider a set-up in which the CRP sensor 23 is configured to measure conductivity, CF1 comprises one or more electrolytes, and CF2 is a glucose solution. The glucose solution has a conductivity that is much smaller than the electrolyte concentrate. FIG. 3 is a graph of measured conductivity, given by the sensor signal S1, during steps 202A-202D in FIG. 2B. During step 202A, as the first test mixture is pumped to the CRP sensor 23, the measured conductivity rises quickly and stabilizes at CRP1. Step 202B is suitably performed during a time period Δt1 when the measured conductivity is stable. During step 202C, as CF2 added to the first test mixture to form the second test mixture, the measured conductivity decreases slightly and stabilizes at CRP2, which is discriminable from CRP1. Step 202D is suitably performed during a time period Δt2 when the measured conductivity is stable. The skilled person understands that CRP2 may be converted into a concentration value of the glucose solution, CF2, when CRP1 is known as well as the relative amounts of base fluid, CF1 and CF2 in the second test mixture. It may be noted that the difference between CRP2 and CRP1 may be partly the result of a dilution effect, if the second test mixture is generated by adding CF2 to the first test mixture. However, provided that the added amount of CF2 is known, the dilution effect may be accounted for when determining the concentration value of CF2 based on CRP2 in relation to CRP1. However, it also possible to generate the second test mixture to eliminate the dilution effect. For example, in the FPA of FIG. 7A (below), an addition of an amount of CF2 will automatically reduce the amount of base fluid by a corresponding amount, thereby eliminating the dilution effect.

If the CRP values of both CF1 and CF2 are significant, for example if conductivity is measured for CF1, CF2 that both include one or more electrolytes, the second test mixture in FIG. 2B may instead be generated as a mixture of the base fluid and CF2. This will still provide the benefit of allowing the CRP sensor 23 to have a relative narrow detection range of CRP values.

The method 200 of FIG. 2A may further comprise a validation phase, implemented by step 208, which may be performed one or more times during a PP or between PPs. The validation phase may be performed to validate the accuracy of the concentration calculation by the method 200 and determine a respective updated ECF for use in the subsequent execution of the method 200. In FIG. 2A, the method proceeds from step 208 to step 204, to perform another PP, in which the FPA 20 is operated to mix the base fluid with CF1, CF2 in proportions that are determined by use of the respective updated ECF from step 208.

In some embodiments of step 208, the control device 30 operates the FPA 20 to pump a concentrate (optionally mixed with base fluid) to the CRP sensor 23, and determines an updated concentration of the concentrate based on the sensor signal S1 from the CRP sensor 23 during the validation phase. The control device 30 then operates to update the respective ECF for the concentrate based on the start concentration (c0) of the concentrate from step 203 and the updated concentration. In some embodiments, the validation phase is performed for each individual concentrate, to derive an updated ECF for each concentrate. However, as will be demonstrated below, it may be possible to use the same ECF to calculate the current concentration in more than one container, at least if the containers are similar or identical. This also means that it may be sufficient to perform the validation procedure for one concentrate only.

FIG. 2C is a flow chart of an example validation procedure that may be performed in step 208 of FIG. 2A to implement a validation phase (VP). In the illustrated example, the validation procedure is performed to validate the concentration calculations for CF1 in container 15 (FIG. 1C). In step 221, the FPA is operated to generate a mixture (“validation mixture”) of the base fluid and CF1, at a third mixing ratio MR3, which may or may not be equal to the first mixing ratio MR1 used in step 202A (FIG. 2B).

The calculations in subsequent step 224 may be facilitated when MR3 equals MR1. In step 221, the FPA is operated to pump the validation mixture to the CRP sensor 23. In step 222, the control device obtains a CRP value, CRP3, for the validation mixture from the sensor signal S1. In step 223, the control device determines an updated concentration of CF1 in the container 15, based on CRP3 and MR3. In step 224, the control device updates the ECF based on the start concentration (c0) of CF1 from step 203 (FIG. 2A), the updated concentration of CF1, the reference time point (t0) and a validation time point associated with the validation phase. The validation time point may be any time point during the validation phase.

The updating by step 224 is exemplified in FIG. 4, which schematically represents CCFs for CF1 in the container 15. FIG. 4 presumes that the FPA is operated to generate medical fluid on demand, during on-going treatment. The ordinate in FIG. 4 designates treatment sessions (TMT #), which are separated in time. It is assumed that a new container 15 is installed before TMT #1 is started. CCF1 designates a CCF given by an ECF stored in memory (cf. FIG. 1C). For example, CCF1 may be given by Eq. (6). To facilitate the following discussion, CCF1 is drawn as a linear function of time: CCF1=NC1+α1·t, with NC1 being the nominal concentration of CF1, and α1 being the rate of concentration change. The nominal concentration is typically the default or target concentration of CF1 during manufacture and may be given by a label on the container 15. The nominal concentration is thus the expected and most probable concentration of CF1 when the container 15 is installed. As indicated, an initialization phase (IP) is performed before TMT #1, resulting in an estimated start concentration (c0) of CF1 (cf. step 203). In the illustrated example, there is a difference OFF1 between NC1 and c0. Before the first production phase is started, CCF1 is adjusted to match the start concentration, resulting in CCF1′. As seen, CCF1′ is formed by shifting CCF1 by OFF1: CCF1′=c0+α1·t. This adjustment is applicable irrespective of how CCF1 is defined. During the production phases of TMT #1 and TMT #2, CCF1′ is used to estimate the current CF1 concentration in the container 15. A validation phase (VP) is performed in advance of TMT #3, resulting in a current CF1 concentration, cc″ (cf. step 223). At this time, the CCF1′ yields a current CF1 concentration, cc′. There is thus a deviation, OFF2, between cc′ and cc″ at the validation time point. In the illustrated example, the CCF1′ has apparently underestimated the evaporative loss from CF1 causing cc″ to exceed cc′. The ECF is then updated in step 224, resulting in CCF1″, which is then used to estimate the current CF1 concentration in the container 15 during the production phases of TMT #3-TMT #6. In the illustrated example, CCF1″ is formed by both shifting CCF1′ and changing its slope.

In some embodiments of step 224, the control device adjusts one or more parameters of the ECF to form an updated ECF that results in an aggregated water loss from the reference time point to the validation time point that matches the difference in water content of the CF1 between these time points. The difference in water content is calculated based on the start concentration and the updated concentration, corresponding to c0 and cc″ in FIG. 4.

The validation phase may be performed more than once. For example, another VP may be performed before start of TMT #5 or #TMT6 in FIG. 4.

It is realized that the validation phase provides feedback on the accuracy of the CCF currently in use, and thereby enables the control device to adapt the CCF to improve the accuracy of the calculated concentration. The adaptation of CCF may be made as an overall adjustment of the evaporation rate (dm/dt) or by adapting the dependence of the ECF on one or more input variables, such as volume (V), humidity (H), or temperature (T). In step 224, the control device compares the calculated concentration change between two points in time with an experimentally determined concentration change between those two points in time. The ECF may be updated in different ways based on this comparison. In a first example, if the deviation cannot be explained by the available input variables, an overall factor may be applied to decrease or increase the evaporation rate (dm/dt). Such unexplained deviations may be caused by an input variable that is not measured. For example, air movement from ventilation or the like may have an unknown influence on the evaporation rate. This also means that even if the ECF does not include temperature (T) and/or humidity (H) as input variables, the updated ECF may still account for the influence of these variables through the validation phase. In a second example, the control device may perform multiple tests at different values of the input variables, and modify the dependence of the ECF on one or more input variables based on the outcome of the tests. The multiple tests and the modification of the ECF may be performed in accordance with conventional procedures for design of experiments (DOE). The multiple tests may be performed during one in-use period or over plural in-use periods.

The present Applicant has surprisingly found that one and the same ECF may be used to estimate the concentration in containers that contain different concentrates. This means that the updated ECF that is determined for CF1 in container 15 by the validation procedure in FIG. 2B may not only be used to calculate the concentration of CF1 in the container 15, but also to calculate the concentration of CF2 in container 16. This will reduce both complexity and duration of the validation phase. Often, the amounts of concentrates that are directed to the CRP sensor during the validation phase are discarded, by being directed to drain. By limiting the validation phase to one concentrate, the overall consumption of concentrates is reduced, which is both environmentally sound and cost effective.

The skilled person understands that even if the same ECF is used, the concentration calculation functions (CCFs) are likely to differ between the containers 15, 16, since the starting concentrations of CF1 and CF2, as well as the momentary volumes of CF1 and CF2, are likely to differ.

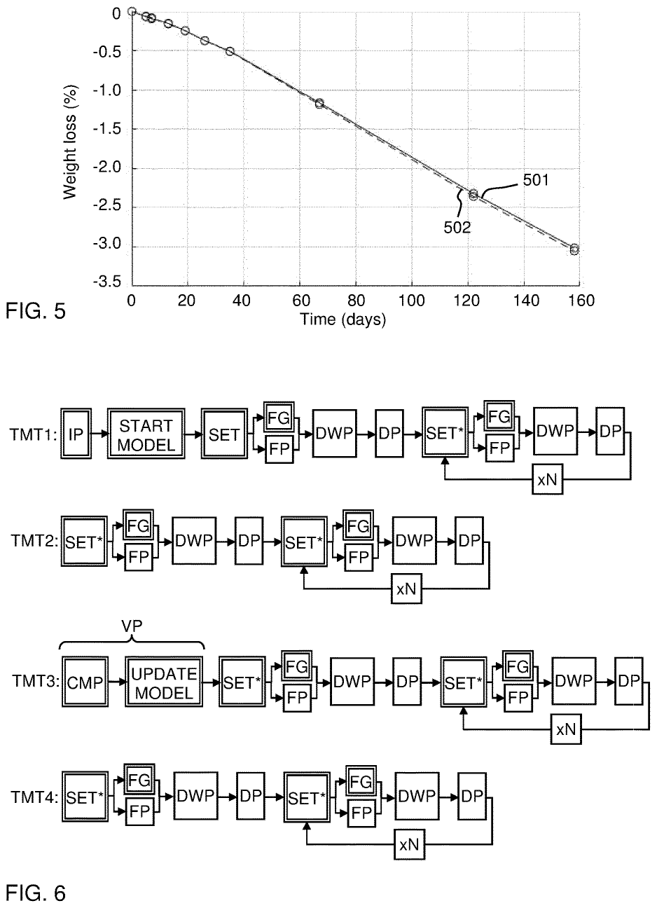

The possibility to use the same ECF for containers with different concentrates is demonstrated by experimental data in FIG. 5. The experimental data is obtained for a first container containing an electrolyte concentrate (1 L PVC bag, 20× Dianeal), and for a second container containing a glucose concentrate (1 L PVC bag, 50% glucose). The containers were hung in free air without over-pouch for 159 days. At selected time points, the containers were weighed and the weight loss relative to the respective initial weight was calculated. In FIG. 5, curves 501, 502 represent the relative weight loss of the first container and the second container, respectively. As seen, the relative water loss is effectively the same for the two containers. It can be shown that the result in FIG. 5 is applicable to all concentrates that are conventionally used for producing medical fluid for use in dialysis therapy.

In the specific example of an electrolyte concentrate and a glucose concentrate, the skilled person realizes that is may be beneficial to perform the validation phase for the electrolyte concentrate instead of the glucose concentrate. The electrolyte concentrate is an ionic solution, which has a high electrical conductivity. Thus, a conductivity sensor may be used in the validation phase to obtain a highly accurate conductivity measurement for the validation mixture. Conductivity sensors are relatively cheap and accurate measurement devices that are commonly used in dialysis machines. It is understood that the combination of electrolyte concentrate and conductivity sensor is likely to result in a high accuracy of the updated ECF. While it is possible to estimate the concentration of a glucose concentrate by use of the technique in FIG. 3, such measurement consumes larger quantities of both concentrates and results in lower measurement accuracy. In summary, by determining the ECF for an electrolyte concentrate, it is possible to decrease concentrate waste and time to therapy, as well as provide a more accurate CCF for the glucose concentrate concentration over the in-use period. The foregoing is equally applicable to concentrates that contain other osmotic agents than glucose.

Reverting to FIG. 2A, it is possible to repeat the initialization phase (IP) during the in-use period to track how the concentration changes in the containers 15, 16 over time due to evaporative loss. However, the amounts of concentrates that are directed to the CRP sensor during the IP are typically wasted and each IP thus increases the monetary and environmental cost of treatment. It is therefore of interest to reduce the number of IPs. The concentration calculations will reduce the need to perform additional IPs after the initial IP, but it is certainly possible to perform at least one additional IP during the in-use period.

FIG. 6 illustrates a sequence of four consecutive sessions of APD therapy, designated TMT1-TMT4, one session per day. Each session comprises 1+N fluid exchange cycles, with N>0. The session is performed by a therapy system 10, for example a cycler (FIG. 1A), by use of dialysis fluid generated on-demand by an FPA 20 (FIG. 1A). The boxes in FIG. 6 represent procedures performed by the control device 30 in relation to the FPA (double line boxes) and the cycler (single line boxes). It is assumed that new prefilled containers 15, 16 are installed on the FGA 20 in preparation of TMT1. TMT1 starts by an initialization phase, IP (steps 202-203). When IP is completed, concentration calculations are started (START MODEL). The concentration calculations are performed by use of a concentration calculation function (CCF) for each container 15, 16. The CCFs are defined by use of an ECF retrieved from memory (step 201) and the start concentrations for CF1, CF2 determined in IP. Then, a set-up procedure (SET) is performed to determine settings for the FPA to generate dialysis fluid with a target composition and a target flow rate. In SET, a mixing ratio between base fluid, CF1 and CF2 is determined to achieve the target composition of the dialysis fluid, and set values for pumping speeds are determined to achieve this mixing ratio and the target flow rate of dialysis fluid to the cycler. After SET, the FPA is operated to generate dialysis fluid (FG), while the cycler is operated to perform a fill phase (FP). Then, the cycler is operated to perform a dwell phase (DWP) and a drain phase (DP) in sequence. During DWP and DP, no dialysis fluid is generated by the FPA. The sequence of FP, DWP and DP forms a fluid exchange cycle. TMT1 comprises N additional cycles. In the illustrated example, an updating procedure (SET*) is performed in advance of each additional cycle to determine updated settings for the FPA. SET* corresponds to steps 206-207 in FIG. 2A and involves estimating the current concentrations of CF1, CF2 in containers 15, 16, and determining an adjusted mixing ratio to achieve the target composition of the dialysis fluid. Thus, SET* compensates for an estimated evaporative loss from containers 15, 16. The updated settings are then used during FG in the subsequent fluid exchange cycle. TMT2 is started with an updating procedure (SET*) to determine updated settings for the FPA. Again, SET* compensates for an estimated evaporative loss from containers 15, 16. SET* is then repeated before each fluid exchange cycle during TMT2. In the illustrated example, TMT3 is started by a validation phase (VP). A concentration measurement procedure (CMP) is performed in accordance with steps 221-223 in FIG. 2C, whereupon the ECF is updated (MODEL UPDATE) in accordance with step 224. For example, the concentration of the electrolyte concentrate CF1 may be measured in CMP. Before the first fluid exchange cycle is started, an updating procedure (SET*) is performed to determine updated settings for the FPA, based on the current measured concentration of CF1 and a current concentration of CF2, which is calculated by use of the updated ECF. TMT3 then proceeds similar to TMT1, and is followed by TMT4, which proceeds similar to TMT2. It is understood that the updated ECF from UPDATE MODEL is used in each subsequent SET* during TMT3 and TMT4 to calculate current concentrations of CF1 and CF2 for determination of updated settings for the FPA.

FIG. 6 is merely given as an example and many variations are possible. For example, SET* need not be performed before each fluid exchange cycle, but may be performed less frequently. It is also conceivable that, depending on dialysis therapy, SET* is performed at one or more time points during the fluid generation procedure (FG), resulting in a seamless adjustment of the mixing ratio during fluid generation. FIG. 7A is a schematic diagram of an FPA configured for on-demand generation of medical fluid. The FPA comprises a chassis 40, which exposes inlet connectors 41A, 42A for releasable connection of containers 15, 16 by connectors 15A, 16A. A main line 43 is defined in the FPA to extend from a source 14 of base fluid to a connection point 48, for example an outlet connector, for connection to a therapy system (not shown). The source 14 may be a tank/container or a separate supply device. The inlet connectors 41A, 42A are fluidly connected to line 43 by a respective supply line 41, 42. A concentrate pump 41B, 42B is arranged in respective line 41, 42, and a main pump 43B is arranged in line 43 downstream of pumps 41B, 42B. The pumps 41B, 42B are operable to dose CF1, CF2 into line 43, and pump 43B is operable to define the flow rate of medical fluid to the outlet connector 48. A mixing device 44 is arranged in line 43 downstream of pump 43B. The mixing device 44 may be a chamber designed to enhance mixing. Further, a heater 46 and a temperature sensor 47A are arranged in line 43 and operable to heat the medical fluid to a target temperature. A conductivity sensor 23 is arranged in line 43 downstream of the mixing device 44. In the illustrated example, the conductivity sensor 23 comprises a temperature sensor 47B, which allows the sensor 23 to output a sensor signal (S1 in FIG. 1C) with temperature-compensated conductivity values.

The FPA in FIG. 7A is operated to generate medical fluid by controlling the speeds of pumps 41B, 42B, 43B. Thus, the above-mentioned mixing ratio corresponds to a relation between the speeds of pumps 41B, 42B, 43B. The speed of pump 43B is set to produce a target flow rate of medical fluid, and the speeds of pumps 41B, 42B are set to generate a target composition of the medical fluid, in accordance with the mixing ratio.

The FPA in FIG. 7A may be operated to perform the initialization phase (IP) as shown in FIG. 4. In accordance with the embodiment in FIG. 2B, pumps 41B, 43B are first operated with predefined speeds that correspond to the first mixing ratio (step 211), and a first conductivity value (CRP1) is measured when the sensor signal S1 has stabilized (step 212). The speeds of pumps 41B, 43B are maintained and pump 42B is started and operated with a speed in a predefined relation to the speed of pump 41B, resulting in the second mixing ratio (step 213). A second conductivity value (CRP2) is measured when the sensor signal S1 has stabilized (step 214). Assuming that CF2 is a glucose solution, the speed of pump 42B will be significantly higher than during generation of medical fluid, to achieve a conductivity change that is sufficient for accurate concentration determination. Then, the start concentration of CF1 is calculated based on the first conductivity value and the first mixing ratio, and the start concentration of CF2 is calculated based on the second mixing ratio, the first conductivity value, and the difference between the first and second conductivity values.

FIG. 7B is a schematic diagram of an FGA configured for batch generation of medical fluid. Many elements are common to and assigned the same reference numerals as elements in FIG. 7A. These elements are not described further. The pumps 41B, 42B in FIG. 7A are replaced by on/off valves 41C, 42C. An on/off valve 43C′ is arranged in the main line 43 between pump 43B and the outlet connector 48. The conductivity sensor 23 is arranged upstream of pump 43B. A first recirculation line 51 is connected to line 43, between pump 43B and valve 43C′, and extends to a mixing tank 44′. A second recirculation line 52 extends from the mixing tank 44′ to line 43 upstream of the junctures of lines 41, 42 on line 43. An on/off valve 43C is arranged in line 43 upstream of the juncture of line 52 on line 43. On/off valves 52C, 53C are also arranged in lines 51, 52. A further outlet connector 49 for connection to a drain line (not shown) is exposed on the chassis 40 and connected by a fluid line 53 to line 43 between pump 43B and the juncture of line 51 to line 43.

The FPA in FIG. 7B is operated to generate medical fluid by sequentially admitting base fluid, CF1 and CF2 into the mixing tank 44′, in any order, while pump 43B is operating. In one example, valves 43C, 51C are first opened to pump a first target amount of base fluid into the mixing tank 44′. The first target amount may be metered volumetrically if pump 43B is a volumetric pump. Alternatively, the target volume may be metered by use of a flow meter (not shown) and/or based on the weight of the mixing tank 44′. Then, valve 43C is closed and valve 41C is opened to pump a second target amount CF1 to the mixing tank 44′. Valve 41C is closed and valve 52C is opened so that the mixture is circulated in a circulation loop, which is formed by the mixing tank 44 and lines 51, 52, 43, until the conductivity measured by sensor 23 is stable. Then, valve 42C is opened and valve 52C is closed to pump a third amount of CF2 to the mixing tank 44′. The valve 42C is closed and valve 52C is opened so that the mixture is circulated through the circulation loop. When sensor 23 measures a stable conductivity, pump 43B is stopped and valves 51C, 52C are closed. At this time, since the first, second and third amounts correspond to a predefined mixing ratio, the mixing tank 44′ contains the medical fluid. To output the medical fluid via connector 48, the valves 43C′, 51C are opened.

The FPA in FIG. 7B is operated in a similar way during the initialization phase (IP), albeit with smaller amounts of base fluid, CF1, and CF2. Further, the first conductivity value is measured (step 212) when the mixture of base fluid and CF1 is formed in the circulation loop (step 211), and the second conductivity value is measured (step 214) when the mixture of base fluid, CF1 and CF2 is formed in the circulation loop (step 213). Then, valve 53C is opened and pump 43B is operated to pump the mixture to drain through outlet connector 49.

Any commercially available concentrate or combination of two or more concentrates may be used to generate the medical fluid. In some embodiments, medical fluid for use in PD is generated by mixing at least one concentrate with water. Example compositions of PD concentrates, to be mixed with water individually or in combination, are disclosed in US2018/0021501 and WO2017/193069, which are incorporated herein by reference. In a non-limiting example, first and second PD concentrates are mixed with water to generate a medical fluid equivalent to Dianeal PD solution, with the first PD concentrate having a dilution factor of 20-30 and containing lactate, sodium, magnesium, and calcium, and the second PD concentrate containing 40%-70% glucose.

In some embodiments, medical fluid for use in EC blood therapy of CKD patients is generated by mixing a single concentrate with water at a dilution ratio of 10-50 by volume. In a non-limiting example, the single concentrate comprises lactate, sodium, potassium, calcium, magnesium, glucose and chloride. Such a concentrate is, for example, commercially available for the PureFlow SL system from NxStage.

Alternatively, the medical fluid may be generated by mixing two concentrates with water. For example, a bicarbonate (B) concentrate and an acid (A) concentrate may be mixed with water at a dilution ratio of 10-50. The bicarbonate concentrate is also known as “base concentrate” and need not contain only bicarbonate. A and B concentrates are commercially available and well-known in the art. In a non-limiting example, the B concentrate comprises bicarbonate, and the A concentrate comprises sodium, potassium, calcium, magnesium, glucose, acetate and chloride. In some A concentrates, acetate is replaced or supplemented by another acid, for example lactate, citrate or hydrochloric acid. In the BiCart Select® system from Baxter, dialysis fluid is generated by mixing three different concentrates, namely a bicarbonate concentrate (BiCart®), a sodium chloride concentrate (SelectCart®), and a concentrate with acid and other electrolytes (SelectBag®). In some embodiments, medical fluid for CRRT treatment of AKI patients is generated by mixing at least one concentrate with water. In a non-limiting example, such a medical fluid comprises bicarbonate, sodium, potassium, calcium, magnesium, phosphate, glucose, acetate and chloride. In one example, a base concentrate and an electrolyte concentrate may be mixed with water to form the medical fluid. For example, the base concentrate may be an alkaline bicarbonate solution, and the electrolyte concentrate may be an acidic glucose-based electrolyte solution.

While the subject of the present disclosure has been described in connection with what is presently considered to be the most practical embodiments, it is to be under-stood that the subject of the present disclosure is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results.

1-24. (canceled)

25. A control device for operating an apparatus for producing a medical fluid for use in dialysis therapy, said control device being configured to operate, in a respective production phase, the apparatus to generate a mixture of a base fluid and one or more concentrate fluids, and produce the medical fluid based on the mixture, wherein each of the one or more concentrate fluids is stored in a respective container, said control device being further configured to:
operate the apparatus, in an initialization phase, to pump the one or more concentrate fluids to a sensor device for measuring a composition-related parameter,
determine, based on an output signal of the sensor device during the initialization phase, a reference concentration of the respective concentrate fluid at a reference time point, and
estimate, by use of the reference concentration and at least one calculation function for calculation of evaporative loss of water from the respective container over time, a momentary concentration of the respective concentrate fluid as a function of time,
wherein the control device is configured to, at one or more time points before or during the respective production phase and based on the momentary concentration, adjust a mixing ratio between the base fluid and the one or more concentrate fluids in the mixture so as to produce the mixture with a target composition.

26. The control device of claim 25, which is configured to calculate, by use of the at least one calculation function, an aggregation of the evaporative loss of water from the respective container, said aggregation being made from the reference time point to a selected time point, wherein the control device is further configured to estimate the momentary concentration of the respective concentrate fluid at the selected time point by compensating the reference concentration of the respective concentrate fluid by the aggregation of the evaporative water loss from the respective container.

27. The control device of claim 25, which is further configured to perform a validation procedure, which comprises:
operating the apparatus to pump at least one concentrate fluid among the one or more concentrate fluids to the sensor device, and
determining, based on an output signal of the sensor device, an updated concentration of the at least one concentrate fluid,
wherein the control device is further configured to update the at least one calculation function based on the reference concentration of the at least one concentrate fluid, the updated concentration of the at least one concentrate fluid, the reference time point and a validation time point being associated with the validation procedure.

28. The control device of claim 25, wherein the one or more concentrate fluids comprises a first concentrate fluid in a first container and a second concentrate fluid in a second container.

29. The control device of claim 28, which is configured to, during the initialization phase:
operate the apparatus to generate a first test mixture of the base fluid and the first concentrate fluid, at a first mixing ratio, and pump the first test mixture to the sensor device,
obtain a first sensor value for the first test mixture from the output signal of the sensor device,
operate the apparatus to generate a second test mixture of at least the base fluid and the second concentrate fluid, at a second mixing ratio, and pump the second test mixture to the sensor device, and
obtain a second sensor value for the second test mixture from the output signal of the sensor device.

30. The control device of claim 29, which is configured to determine, based on the first and second mixing ratios and the first and second sensor values, the reference concentration of the first concentrate fluid and the reference concentration of the second concentrate fluid.

31. The control device of claim 29, wherein the at least one calculation function comprises a first calculation function for the first container, and wherein the control device is further configured to perform a validation procedure, wherein the validation procedure comprises:
operating the apparatus to generate a further mixture of the base fluid and the first concentrate fluid, at a further mixing ratio, and pump the further mixture to the sensor device,
obtaining a further sensor value for the further mixture from the output signal of the sensor device, and
determining, based on the further sensor value and the further mixing ratio, an updated concentration of the first concentrate fluid,
wherein the control device is further configured to update the first calculation function based on the reference concentration of the first concentrate fluid, the updated concentration of the first concentrate fluid, the reference time point and a validation time point associated with the validation procedure.

32. The control device of claim 31, which is configured to update the first calculation function by adjusting one or more parameters of the first calculation function so that that the first calculation function results in an aggregated water loss from the reference time point to the validation time point that matches a difference in water content between the reference concentration of the first concentrate fluid and the updated concentration of the first concentrate fluid.

33. The control device of claim 31, which is configured to, before or during the respective production phase after the validation procedure, estimate the momentary concentration of the first concentrate fluid in the first container as a function of time by use of the thus-updated first calculation function.

34. The control device of claim 33, which is configured to, before or during the respective production phase after the validation procedure, estimate the momentary concentration of the second concentrate fluid in the second container as a function of time by use of the thus-updated first calculation function.

35. The control device of claim 28, wherein the first concentrate fluid comprises one or more electrolytes, and wherein the second concentrate fluid comprises an osmotic agent.

36. The control device of claim 25, wherein the reference concentration and the momentary concentration represent the concentration of a representative solute in the respective concentrate fluid.

37. The control device of claim 25, wherein the base fluid is purified water.

38. The control device of claim 25, wherein the at least one calculation function is configured to quantify a momentary evaporative loss of water from the respective container over time.

39. The control device of claim 38, which is configured to convert the momentary evaporative loss of water from the respective container over time into the momentary concentration of the respective concentrate fluid as a function of time, based on the reference concentration of the respective concentrate fluid and based on a momentary amount of the respective concentrate fluid in the respective container over time.

40. The control device of claim 25, which is configured to determine the amount of concentrate fluid within the respective container over time during the respective production phase.

41. The control device of claim 25, wherein the calculation function is a function of at least one of: a physical property of the respective container, a humidity in ambient air at the respective container, a temperature of the concentrate fluid within the respective container, a partial vapor pressure of water in the concentrate fluid within the respective container, or an amount of concentrate fluid in the respective container.

42. The control device of claim 25, wherein the composition-related parameter represents electrical conductivity.

43. An apparatus for producing a medical fluid for use in dialysis therapy, said apparatus comprising:
an inlet for a base fluid,
an inlet for a respective concentrate fluid,
a mixing arrangement operable to mix the base fluid with the respective concentration fluid,
a fluid distribution arrangement operable to selectively supply the base fluid and the respective concentration fluid to the mixing arrangement,
a sensor device for measuring a composition-related parameter, and
a control device configured to operate the apparatus for producing the medical fluid, said control device being configured to operate, in a respective production phase, the apparatus to generate a mixture of a base fluid and one or more concentrate fluids, and produce the medical fluid based on the mixture, wherein each of the one or more concentrate fluids is stored in a respective container, said control device being further configured to:
operate the apparatus, in an initialization phase, to pump the one or more concentrate fluids to a sensor device for measuring a composition-related parameter,
determine, based on an output signal of the sensor device during the initialization phase, a reference concentration of the respective concentrate fluid at a reference time point, and
estimate, by use of the reference concentration and at least one calculation function for calculation of evaporative loss of water from the respective container over time, a momentary concentration of the respective concentrate fluid as a function of time,
wherein the control device is configured to, at one or more time points before or during the respective production phase and based on the momentary concentration, adjust a mixing ratio between the base fluid and the one or more concentrate fluids in the mixture so as to produce the mixture with a target composition.

44. A computer-implemented method of operating an apparatus to produce a medical fluid for use in dialysis therapy by mixing a base fluid with one or more concentrate fluids, wherein each of the one or more concentrate fluids is stored in a respective container, said method comprising:
operating the apparatus, in an initialization phase, to pump the one or more concentrate fluids to a sensor device for measuring a composition-related parameter;
determining, based on an output signal of the sensor device during the initialization phase, a reference concentration of the respective concentrate fluid at a reference time point;
operating, in a respective production phase, the apparatus to generate a mixture of a base fluid and one or more concentrate fluids at a mixture ratio and to produce the medical fluid based on the mixture;
estimating, by use of the reference concentration and at least one calculation function for calculation of evaporative loss of water from the respective container over time, a momentary concentration of the respective concentrate fluid as a function of time; and
adjusting, at one or more time points before or during the respective production phase and based on the momentary concentration, the mixing ratio between the base fluid and the one or more concentrate fluids in the mixture so as to produce the mixture with a target composition.
